(19) United States
(12) Patent Application Publication
(10) Pub. No.: US 2019/0388516 A1
(43) Pub. Date: Dec. 26, 2019

(54) HEMOSTATIC PRODUCTS

(71) Applicant: St. Teresa Medical, Inc., Eagan, MN (US)

(72) Inventors: Timothy Floyd, Eagan, MN (US); Philip A. Messina, Eagan, MN (US)

(21) Appl. No.: 16/559,359

(22) Filed: Sep. 3, 2019

Related U.S. Application Data
(63) Continuation-in-part of application No. 15/729,934, filed on Oct. 11, 2017.
(60) Provisional application No. 62/406,783, filed on Oct. 11, 2016.

(51) Int. Cl. A61K 38/36; A61K 38/48; A61K 47/02; A61K 9/50; A61K 47/36

(52) U.S. Cl. CPC A61K 38/363; A61K 38/4833; A61K 47/36; A61K 9/5015; A61K 47/02

(57) ABSTRACT

A hemostatic product that includes a dry fibrinogen mixture, a dry thrombin mixture and a biologically tolerable liquid. The dry fibrinogen mixture includes fibrinogen and at least one fibrinogen stabilizer. The dry thrombin mixture includes thrombin and at least one thrombin stabilizer. The biologically tolerable liquid is mixed with the dry fibrinogen mixture and the dry thrombin mixture to form the hemostatic product.

### REFERENCE TO RELATED APPLICATION

This application is a continuation-in-part of U.S. application Ser. No. 15,729,934, filed on Oct. 11, 2017, and claims priority to Provisional Applic. No. 62/406,783, filed on Oct. 11, 2016, the contents of which are incorporated herein by reference.

### FIELD OF THE INVENTION

The invention relates generally to products having hemostatic characteristics. More particularly, the invention relates to hemostatic products that are readily applied to wounds.

### BACKGROUND OF THE INVENTION

The body's natural response to stem bleeding from a wound is to initiate blood clotting via a complex process known as the coagulation cascade. The cascade involves two pathways that ultimately lead to the production of the enzyme thrombin, which catalyzes the conversion of fibrinogen to fibrin.

Fibrin is then cross-linked to form a clot, resulting in hemostasis. For wounds that are not severe, and in individuals that have no countervening conditions, the body is usually able to carry out this process efficiently in a manner that prevents excessive loss of blood from the wound. However, in the case of severe wounds, or in individuals in whom the clotting mechanism is compromised, this may not be the case.

For such individuals, it is possible to administer components of the coagulation cascade, especially thrombin and fibrinogen, directly to the wound to bring about hemostasis. Bandaging of bleeding wounds is also a usual practice, in part to isolate and protect the wounded area, and also to provide a means to exert pressure on the wound, which can also assist in controlling bleeding.

Bowlin et al., U.S. Patent Publication No. 2011/0150973, discloses a method of delivering one or more agents of interest to a location of interest. The method includes applying or delivering to a location of interest a hemostatic product. The hemostatic product includes electrospun dextran fibers that dissolve upon contact with liquid. The hemostatic product also includes one or more agents of interest associated with said electrospun dextran fibers. Applying or delivering results in dissolution of the electrospun dextran fibers in liquid at the location of interest to thereby release the one or more agents of interest into the liquid.

There is an ongoing need to provide improved methods and means to initiate blood clotting in wounds to stop or at least slow blood loss. In particular, there is an ongoing need to improve the capability to readily promote hemostasis in severe wounds in a facile manner, especially under circumstances where immediate treatment by medical personnel is limited or unavailable.

### SUMMARY OF THE INVENTION

An embodiment of the invention is directed to a hemostatic product. The dry fibrinogen mixture includes fibrinogen and at least one fibrinogen stabilizer. The dry thrombin mixture includes thrombin and at least one thrombin stabilizer. The biologically tolerable liquid is mixed with the dry fibrinogen mixture and the dry thrombin mixture to form the hemostatic product.

Another embodiment of the invention is directed to a method of using a hemostatic product. A dry fibrinogen mixture is prepared that includes fibrinogen and at least one fibrinogen stabilizer. A dry thrombin mixture is prepared that includes thrombin and at least one thrombin stabilizer. The dry fibrinogen mixture and the dry thrombin mixture are mixed with a biologically tolerable liquid to form the hemostatic product. The hemostatic product is applied to a wound to cause hemostasis.

### DETAILED DESCRIPTION OF THE PREFERRED EMBODIMENTS

An embodiment of the invention is directed to a system for providing hemostasis in a person or animal. When the hemostatic product is applied to the injury site, the materials used to fabricate the hemostatic product dissolve to thereby release the materials to the injury site and provide the hemostatic effect.

In some embodiments of the invention, only hemostatic components are utilized and thus after clot formation, there is no need to disturb the clot to remove hemostatic product components, since none remain at the site. The hemostatic product thereby does not leave any residual foreign bodies that elicit foreign body reactions or act as a nidus for infection. Furthermore, the hemostatic product does not contain any xenoproteins, which have the potential of eliciting immune reactions in persons on which the hemostatic product is used.

In other embodiments, as described below, the hemostatic product may include other materials such as support or backing material, which, after initial rapid application of the hemostatic product, may later be removed for further treatment of the wound by conventional methods.

The thrombin and fibrinogen that are used in fabricating the hemostatic product are in forms that are biologically active when they come into contact with blood. Hence, upon dissolution, the thrombin acts on the fibrinogen, converting it to fibrin, which then forms a clot within the wound to thereby staunch the flow of blood.

In certain embodiments, the thrombin and fibrinogen may be derived from human sources. In other embodiments, the thrombin and fibrinogen are salmon thrombin and fibrinogen. Advantages of using salmon as a source of these materials include but are not limited to the lack of concern about transmission of etiologic agents (e.g. viruses) that may occur when human and other mammalian sources of thrombin or fibrinogen (e.g. bovine) are used.

The thrombin and fibrinogen may be utilized in a variety of forms such as liquid, solid and liquid reconstituted from frozen or in powder lyophilized. It is possible that the form of thrombin that is used may be different from the form of fibrinogen that is used.

The components used in fabricating the hemostatic product should be selected to be the same as components found in a living body where the hemostatic product is to be used. Alternatively, the components used in fabricating the hemostatic product are compatible with and readily broken down when the hemostatic product is used on or in a living body. Using such a process minimizes complications associated with components of the hemostatic product not being promptly being broken down as such a process could cause inflammation in the living body. The only thing that remains after the use of the hemostatic product is the clot, which most living bodies are adapted to degrade over time.

A fibrinogen mixture may be prepared by mixing fibrinogen with at least one salt. Mixing the fibrinogen with the at least one salt enhances the stability of the fibrinogen during storage prior to fabricating the hemostatic product as well as after the hemostatic product is fabricated.

In certain embodiments, the dry fibrinogen mixture comprises fibrinogen that is derived from a human source. The fibrinogen may be provided at a concentration of between about 40 percent by weight and about 70 percent by weight. In other embodiments, the fibrinogen is provided at a concentration of between about percent by weight 50 percent by weight and about 60 percent by weight.

In certain embodiments, the at least one salt is sodium chloride. The at least one salt is provided at a concentration of between about 15 percent by weight and about 50 percent by weight. In other embodiments, the at least one salt is provided at a concentration of between about 25 percent by weight and about 35 percent by weight.

The at least one salt may also include trisodium citrate that is provided at a concentration of between about 10 percent by weight and about 30 percent by weight. In other embodiments, the trisodium citrate is provided at a concentration of between about 15 percent by weight and about 25 percent by weight.

A dry thrombin mixture may be prepared by mixing thrombin with at least one salt and at least one buffer. Mixing the thrombin with the at least one salt and the at least one buffer enhances the stability of the thrombin prior to fabricating the hemostatic product as well as after the hemostatic product is fabricated.

In certain embodiments, the thrombin comprises thrombin that is derived from a human source. The thrombin may be provided at a concentration of between about 0.5 percent by weight and about 2.5 percent by weight. In other embodiments, the thrombin is provided at a concentration of between about 1.0 percent by weight and about 1.5 percent by weight.

In certain embodiments, the at least one salt is sodium chloride. The at least one salt is provided at a concentration of between about 50 percent by weight and about 70 percent by weight. In other embodiments, the at least one salt is provided at a concentration of between about 60 percent by weight and about 65 percent by weight.

In certain embodiments, the at least one buffer is bis(2-hydroxyethyl)amino-tris(hydroxymethyl)methane. The at least one buffer is provided at a concentration of between about 15 percent by weight and about 35 percent by weight. In other embodiments, the at least one buffer is provided at a concentration of between about 25 percent by weight and about 35 percent by weight.

The dry thrombin mixture may also include a biologically tolerable polymer. An example of one such biologically tolerable polymer is poly(ethylene glycol). The poly(ethylene glycol) may have a molecular weight of between about 5,000 and about 20,000. In certain embodiments, the poly(ethylene glycol) has a molecular weight of between about 7,000 and about 10,000.

The biologically tolerable polymer is provided at a concentration of between about 5 percent by weight and about 25 percent by weight. In other embodiments, the biologically tolerable polymer is provided at a concentration of between about 8 percent by weight and about 20 percent by weight. In still other embodiments, the concentration of the biologically tolerable polymer is between about 13 percent by weight and about 20 percent by weight.

The components used to fabricate the dry thrombin mixture are mixed together until a substantially homogeneous mixture is prepared. The mixing process should be sufficiently gentle so that undue stress is not placed on the thrombin particles as such stress could impact the efficacy of the thrombin during the use of the hemostatic product.

Once the mixing of the dry fibrinogen mixture and the dry thrombin mixture has been completed, the dry fibrinogen mixture and the dry thrombin mixture may be packaged prior to mixing together and applying to the site at which it is desired to achieve hemostasis. Packaging reduces the exposure of the dry fibrinogen mixture and the dry thrombin mixture to moisture as exposure to moisture can negatively impact the efficacy of the dry fibrinogen mixture and the dry thrombin mixture by causing the proteins to activate before the proteins are needed to achieve hemostasis.

To reduce the moisture concentration in the container, a desiccant may be placed in the container. A person of skill in the art will appreciate that a variety of desiccants may be selected based upon factors such as the initial moisture content of the dry fibrinogen mixture and the dry thrombin mixture.

The dry fibrinogen mixture and the dry thrombin mixture needs to be sterilized. The sterilization is preferably done after the dry fibrinogen mixture and the dry thrombin mixture are packaged.

A variety of techniques may be used to sterilize the dry fibrinogen mixture and the dry thrombin mixture. The selected sterilization technique should minimize denaturation of the fibrinogen and the thrombin. Even while the denaturation of the fibrinogen and the thrombin is minimized, it is anticipated that at least some of the fibrinogen and the thrombin will be denatured during the sterilization process. The denaturation should be less than about 20 percent.

The moisture level in the container can further be reduced by injecting an inert gas into the container. In certain embodiments, the inert gas is nitrogen. The processing reduces the moisture level in the hemostatic mixture container to less that about 6 percent, which is a significant reduction from the initial moisture level of the hemostatic mixture, which is about 11 percent depending on ambient conditions. In other embodiments, the moisture level in the container is less than about 3 percent. Another benefit of the nitrogen injection is that the nitrogen injection displaces oxygen from the container.

Another technique to minimize denaturation of the components in the dry fibrinogen mixture and the dry thrombin mixture is to maintain the dry fibrinogen mixture and the dry thrombin mixture at a low temperature prior to use. The temperature may be less than about 32° F. In other embodiments, the temperature is less than about 0° F. One technique that may be used to maintain the dry fibrinogen mixture and the dry thrombin mixture at the low temperature is placing dry ice adjacent to the dry fibrinogen mixture and the dry thrombin mixture.

Proximate to when it is desired to use the hemostatic product, the dry thrombin mixture and the dry fibrinogen mixture are mixed together to form the hemostatic product. A concentration of the dry fibrinogen mixture in the hemostatic product is between about 20 percent by weight and about 80 percent by weight. A concentration of the dry thrombin mixture in the hemostatic product is between about 20 percent by weight and about 80 percent by weight. In certain embodiments, a biologically tolerable liquid may be used in fabricating the hemostatic product. A concentration of the biologically tolerable liquid in the hemostatic product is up to about 50 percent by weight.

As used herein, the term biologically tolerable liquid encompasses liquids that are suitable for use on or in a human body without experiencing negative side effects. A non-limiting example of one suitable biologically tolerable liquid is water.

The biologically tolerable liquid can thereby be used to provide the hemostatic product with a variety of consistencies based upon the intended use of the hemostatic product. In certain embodiments, the hemostatic product thereby produced could be viewed as a fibrin paste.

In some embodiments, the addition of the biologically tolerable liquid is done at a concentration that facilitates the hemostatic product having a high viscosity. As used herein, the term high viscosity means that the viscosity of the hemostatic product is greater than about 1,000 centipoise. In other configurations, the term high viscosity is between about 10,000 centipoise and about 100,000 centipoise. Forming the hemostatic product with the high viscosity allows the hemostatic product to remain substantially stationary when placed on an area where it is desired to achieve hemostasis. For example, the hemostatic product may be applied to a bleeding site or a site from which cerebrospinal fluid is leaking. It is also possible to use the hemostatic product in conjunction with treating burns to enhance the rate at which the burns heal.

In other embodiments, the hemostatic product may be formed with a relatively low viscosity so that the hemostatic product can be poured on to an area where it is desired to achieve hemostasis. As used herein, the term low viscosity is less than about 1,000 centipoise. In other configurations, the low viscosity is between about 1 centipoise and about 500 centipoise.

The quantity of fibrinogen in the hemostatic product may be adjusted by changing the concentration of the fibrinogen that is used in the dry fibrinogen mixture or the concentration of the dry fibrinogen mixture that is used in the hemostatic product. The quantity of fibrinogen in the hemostatic product is generally in the range of from about 10 milligrams to about 3 grams. In certain embodiments, the amount of fibrinogen in the hemostatic product is between about 20 milligrams to about 1 gram.

The quantity of thrombin in the hemostatic product may be adjusted by changing the concentration of the thrombin that is used in the dry thrombin mixture or the concentration of the dry thrombin mixture that is used in the hemostatic product. The quantity of thrombin in the hemostatic product is generally between about 10 and 10,000 NIH Units. In certain embodiments, the amount of thrombin in the hemostatic product is between about 20 and 6,000 NIH Units.

It is also possible to change the concentration of the fibrinogen and the thrombin through the addition of filler material. This filler material enables at the concentration of the fibrinogen and the thrombin to be changed while maintaining the hemostatic product at a desired viscosity.

In certain embodiments, the filler material is added while the dry fibrinogen mixture and the dry thrombin mixture are being mixed together. In other embodiments, the filler material can also be added after the dry fibrinogen mixture and the dry thrombin mixture are mixed together to form the hemostatic product. In other embodiments, the filler material is added to at least one of the dry fibrinogen mixture and the dry thrombin mixture.

The filler material should be biocompatible such that the filler material does not cause issues when the hemostatic product is used to achieve hemostasis. The filler material should also readily dissolve and either readily breakdown or bioabsorb so that the filler material does not remain after the hemostatic product is used to achieve hemostasis. An example of one material that may be used for the filler material is dextran. The dextran may be provided in a variety of forms, an example of one suitable form is a powder.

In some applications, the fibrinogen and/or the thrombin may be electrosprayed with sucrose to form sugar droplets, which tends to stabilize the fibrinogen and the thrombin and can also “trap” other substances of interest for delivery to the hemostatic product. For thrombin and fibrinogen, in most embodiments, these (or other) active agents are in a finely dispersed dry, particulate or granular form e.g. as a fine powder or dust.

Usually the agents are bioactive agents that have a beneficial or therapeutic effect at the wound site. In one embodiment, the site is a bleeding wound at which it is desired to form a blood clot to stop or slow the bleeding. In this embodiment, the therapeutic substances of interest may include, for example, thrombin and fibrinogen, although other agents active in promoting hemostasis, including but not limited to capscian, may also be included.

In addition, collagen, agents that absorb water, various dry salts that would tend to absorb fluids when placed in contact with e.g. blood; engineered thrombin or thrombin mimics; engineered fibrinogen; agents that cause vasospasm (e.g. ADP, 5-hydroxytryptamine, 5-HT and thromboxane, (TXA-2) to help contract and seal a bleeding vessel, etc. may also be included.

Other components of the clotting cascade may be utilized in conjunction with the fibrinogen and thrombin, for example: tissue factors that are normally only expressed on the surface of damaged cells and that start the normal clotting cascade; serotonin which enhances platelet clumping and promotes vessel constriction; and other agents that are used to replace missing components of the clotting cascade in hemophilia, for example, factor 7 (which activates the so called external extrinsic coagulation cascade) and crude extracts of platelets.

Active agents that function to promote late stages of wound healing may also be included to, for example, facilitate cell migration and remodeling. The incorporation of collagen is an example of such an active agent.

One or more of any of these active agents may be used in the practice of the present invention. The therapeutic agents must be amenable to drying and are associated with the other components of the hemostatic product in the dry state, since liquid may negatively affect at least one of the components used in the hemostatic product. For example, the active agents may be desiccated or lyophilized, or water may be removed by other means.

These additional components may be mixed with the fibrinogen and/or the thrombin prior to these components being mixed together, while these components are being mixed together and/or after these components are mixed together. Such mixing may be accomplished by any of many suitable techniques that are known to those of skill in the art, and will depend in part on the precise form of the substance and the means at hand. For example, for powdered, particulate thrombin and fibrinogen, association may be carried out by sprinkling, shaking, blowing, etc. the agents onto a layer of the excipient or carrier.

In some embodiments of the invention, one or more support structures or support materials incorporated therein may be placed over the hemostatic product that is applied. The support material may be formed from various electrospun materials such as polyglycolic acid (PGA), polylactic acid (PLA), and their copolymers (PLGAs); charged nylon, etc. In one embodiment, the support material is compressed electrospun dextran fibers. By “compressed electrospun dextran fibers,” it is meant that electrospun dextran fibers are compressed together under pressure.

The support material may or may not be soluble in liquid, or may be slowly soluble in liquid, and may or may not be permeable to liquid. Slowly soluble materials include those from which absorbable or dissolving (biodegradable) stitches or sutures are formed, included PGA, polylactic and caprolactone polymers.

In certain embodiments, the support material may dissolve relatively quickly such as less than about 1 hour. In other embodiments, the support material may dissolve within from about 10 days to 8 weeks. In either case, the support material provides the advantage of not having to remove the hemostatic product and risk disrupting the clot.

However, in any case, the support material should not interfere with achieving hemostasis. All such arrangements, shapes, and embodiments of carrier layers and support materials as described herein are intended to be encompassed by the invention.

The invention may also include diagnostic agents that can be used by the treating medical professional to diagnose the nature of the injury. In certain embodiments, the diagnostic agent may change colors to indicate the presence of particular chemicals in the blood or to indicate particular characteristics of the blood. For example, if the patient is currently taking medications that cause thinning of the patient's blood. The diagnostic agents could also change colors to indicate the oxygen and/or glucose level of the blood.

In addition to being used to produce hemostasis in humans, the concepts of the invention may be adapted for use in conjunction with other animals. Examples of such animals on which the invention can be used include dogs and cats.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A method of preparing a hemostatic product comprising:
preparing a dry fibrinogen mixture consisting essentially of mixing fibrinogen and at least one fibrinogen stabilizer;
preparing a dry thrombin mixture consisting essentially of mixing thrombin and at least one thrombin stabilizer; and
mixing the dry fibrinogen mixture and the dry thrombin mixture with a biologically tolerable liquid to form the hemostatic product.

2. The method of claim 1, wherein preparing the dry fibrinogen mixture comprises at least partially coating the fibrinogen with a sugar, wherein preparing the dry thrombin mixture comprises at least partially coating the thrombin with a sugar and wherein the at least one fibrinogen stabilizer and the at least one thrombin stabilizer comprise a sodium chloride.

3. The method of claim 1, wherein a concentration of the dry fibrinogen mixture in the hemostatic product is between about 20 percent by weight and about 80 percent by weight, wherein a concentration of the dry thrombin mixture in the hemostatic product is between about 20 percent by weight and about 80 percent by weight, and wherein a concentration of the biologically tolerable liquid in the hemostatic product is up to about 50 percent by weight.

4. The method of claim 1, and further comprising mixing a filler material into the hemostatic product, wherein the filler material is provided in the hemostatic product at a concentration of between about 5 percent by volume and about 25 percent by volume.

5. The method of claim 4, wherein the filler material is dextran and wherein the biologically tolerable liquid is water.

6. The method of claim 1, wherein the hemostatic product has a viscosity of between about 10,000 centipoise and about 100,000 centipoise.

7. The method of claim 1, wherein the hemostatic product has a viscosity of between about 1 centipoise and about 500 centipoise.

8. A hemostatic product comprising:
a dry fibrinogen mixture comprising fibrinogen and at least one fibrinogen stabilizer;
a dry thrombin mixture comprising thrombin and at least one thrombin stabilizer; and
a biologically tolerable liquid that is mixed with the dry fibrinogen mixture and the dry thrombin mixture to form the hemostatic product.

9. The hemostatic product of claim 8, wherein at least one of the fibrinogen and the thrombin is at least partially coated with a sugar.

10. The hemostatic product of claim 8, wherein the at least one fibrinogen stabilizer and the at least one thrombin stabilizer comprise a salt.

11. The hemostatic product of claim 8, wherein the dry thrombin mixture further comprises a biologically tolerable polymer.

12. The hemostatic product of claim 8, wherein a concentration of the dry fibrinogen mixture in the hemostatic product is between about 20 percent by weight and about 80 percent by weight, wherein a concentration of the dry thrombin mixture in the hemostatic product is between about 20 percent by weight and about 80 percent by weight, and wherein a concentration of the biologically tolerable liquid in the hemostatic product is up to about 50 percent by weight.

13. The hemostatic product of claim 8, and further comprising a filler material, wherein the filler material is dextran that is provided in the hemostatic product at a concentration of between about 5 percent by volume and about 25 percent by volume.

14. The hemostatic product of claim 8, wherein the biologically tolerable liquid is water.

15. The hemostatic product of claim 8, wherein the hemostatic product has a viscosity of between about 10,000 centipoise and about 100,000 centipoise.

16. The hemostatic product of claim 8, wherein the hemostatic product has a viscosity of between about 1 centipoise and about 500 centipoise.

17. A method of using a hemostatic product comprising:
preparing a dry fibrinogen mixture comprising fibrinogen and at least one fibrinogen stabilizer;
preparing a dry thrombin mixture comprising thrombin and at least one thrombin stabilizer;
mixing the dry fibrinogen mixture and the dry thrombin mixture with a biologically tolerable liquid to form the hemostatic product; and
applying the hemostatic product to a wound to cause hemostasis.

18. The method of claim 17, wherein preparing the dry fibrinogen mixture comprises at least partially coating the fibrinogen with a sugar, wherein preparing the dry thrombin mixture comprises at least partially coating the thrombin with a sugar and wherein the at least one fibrinogen stabilizer and the at least one thrombin stabilizer comprise a salt.

19. The method of claim 17, wherein a concentration of the dry fibrinogen mixture in the hemostatic product is between about 20 percent and about 80 percent, wherein a concentration of the dry thrombin mixture in the hemostatic product is between about 20 percent and about 20 percent, and wherein a concentration of the biologically tolerable liquid in the hemostatic product is between about 5 percent and about 50 percent.

20. The method of claim 17, and further comprising mixing a filler material into the hemostatic product, wherein the filler material is provided in the hemostatic product at a concentration of between about 5 percent by volume and about 25 percent by volume, wherein the filler material is dextran and wherein the biologically tolerable liquid is water.
